(19) United States
(12) Patent Application Publication
(10) Pub. No.: US 2018/0218637 A1
(43) Pub. Date: Aug. 2, 2018

(54) METHOD AND ELECTRONIC DEVICE FOR PROVIDING HEALTH CONTENT

(71) Applicant: Samsung Electronics Co., Ltd, Suwon-si (KR)

(72) Inventors: Mi Sun Lee, Anyang-si (KR); Jae Hyeon KANG, Suwon-si (KR); Byoung Tack ROH, Suwon-si (KR); Jeong Hyun PANG, Seongnam-si (KR); Yong Joon JEON, Hwaseong-si (KR); Bo Kun CHOI, Seoul (KR)

(21) Appl. No.: 15/886,215

(22) Filed: Feb. 1, 2018

(30) Foreign Application Priority Data
Feb. 1, 2017 (KR) 10-2017-0014363

(51) Int. Cl. G09B 19/00; G09B 5/02; G16H 20/60; G16H 50/30; G16H 80/00

(52) U.S. Cl. CPC G09B 19/0092; G09B 5/02; G16H 80/00; G16H 50/30; G16H 20/60

(57) ABSTRACT

An electronic device includes a display, a processor, a communication circuit establishing communication with a server, and a memory storing a specified application. The memory stores an instruction that, when executed, causes the processor to output an execution screen of the specified application to the display, in response to a launching of the specified application, to analyze a correlation between a specified health parameter and content included in the execution screen, to generate a specified query message depending on the analysis result, to transmit the query message to the server, to receive a response content of the query message from the server, and to output the response content to at least part of the execution screen.

### CROSS-REFERENCE TO RELATED APPLICATION(S)

This application is related to and claims priority to Korean Patent Application No. 10-2017-0014363 filed on Feb. 1, 2017, the entire disclosure of which is hereby incorporated by reference.

### TECHNICAL FIELD

The present disclosure relates to a method of providing health content and an electronic device performing the same.

### BACKGROUND

Nowadays, there is a growing interest in health. As a result, a variety of medical devices for home use are distributed, a portable electronic device such as a smartphone is also equipped with a GPS module, a heart rate sensor, a pedometer module, or the like. Accordingly, the exercise quantity of a user may be measured by using the devices. Also, devices for the health and fitness are developed as wearable devices to support the user's healthy life in various ways.

### SUMMARY

Various types of devices may measure various parameters (hereinafter refer to as a “health parameter”) indicating the health state or the exercise state of a user. However, there was a limit in providing the diversified and personalized advice to the user, by using personal health information measured by a user terminal or an external device.

Aspects of the present disclosure are to address at least the above-mentioned problems and/or disadvantages and to provide at least the advantages described below. According to this disclosure, it is an object to provide a method capable of providing advice about health by using personal health information, and an electronic device performing the same.

According to certain embodiments of the present disclosure, an electronic device may include a display, a processor, a communication circuit establishing communication with a server, and a memory storing a specified application. The memory may store an instruction that, when executed, causes the processor to output an execution screen of the specified application to the display, in response to a launching of the specified application, to analyze correlations between a specified health parameter and content included in the execution screen, to generate a specified query message depending on the analysis result, to transmit the query message to the server, to receive a response content of the query message from the server, and to output the response content to at least part of the execution screen.

According to certain embodiments of the present disclosure, a health content providing method of an electronic device may include outputting an execution screen of the specified application in response to a launching of the specified application, analyzing correlations between a specified health parameter and content included in the execution screen, generating a specified query message depending on the analysis result, transmitting the query message to a server, receiving a response content of the query message from the server, and outputting the response content to at least part of the execution screen.

According to various embodiments of the present disclosure, health content is provided to a user based on the current health state and/or current activity state of the user. In addition, personalized health content corresponding to each application is provided by interworking with various application programs. Besides, a variety of effects directly or indirectly understood through this disclosure may be provided.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an electronic device, according to various embodiments;

FIG. 2 illustrates a block diagram of the electronic device, according to various embodiments of the present disclosure;

FIG. 3 illustrates a block diagram of a program module, according to various embodiments of the present disclosure;

FIG. 4 illustrates a block diagram of an electronic device according to certain embodiments of the present disclosure;

FIG. 5 illustrates a block diagram of an electronic device according to certain embodiments of the present disclosure;

FIG. 6 illustrates a flowchart of a health content providing method, according to certain embodiments of this disclosure;

FIG. 7 illustrates a view of aspects of a web browser application, according to certain embodiments of this disclosure;

FIG. 8 illustrates aspects of an image viewer application, according to certain embodiments of this disclosure;

FIG. 9 illustrates aspects of a map application, according to certain embodiments of this disclosure; and

FIG. 10 illustrates aspects of an interactive application, according to embodiments of this disclosure.

Throughout the drawings, it should be noted that like reference numbers are used to depict the same or similar elements, features, and structures.

### DETAILED DESCRIPTION

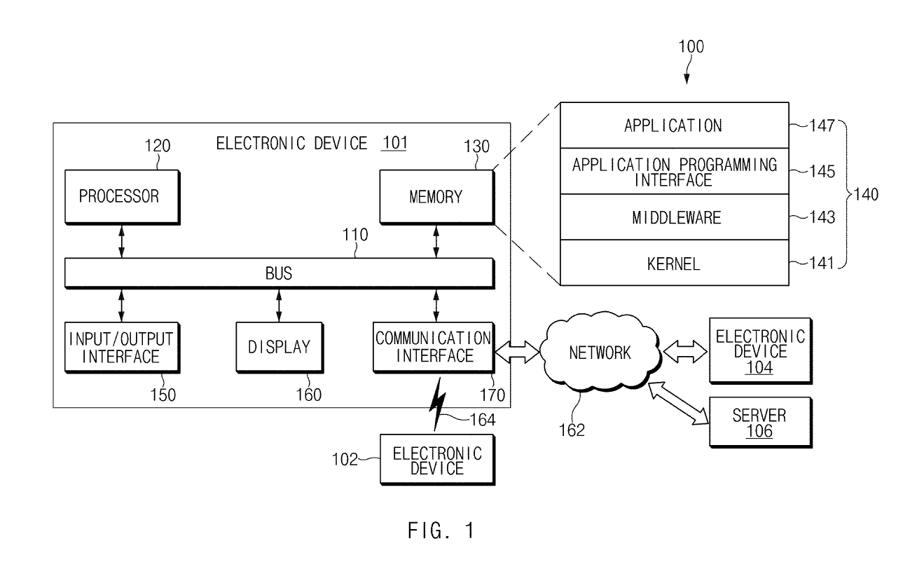

FIGS. 1 through 10, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

Hereinafter, various embodiments of the present invention may be described with reference to accompanying drawings. Accordingly, those of ordinary skill in the art will recognize that modification, equivalent, and/or alternative on the various embodiments described herein can be variously made without departing from the scope and spirit of the present invention. With regard to description of drawings, similar elements may be marked by similar reference numerals.

In this disclosure, the expressions “have”, “may have”, “include” and “comprise”, or “may include” and “may comprise” used herein indicate existence of corresponding features (e.g., elements such as numeric values, functions, operations, or components) but do not exclude presence of additional features.

In this disclosure, the expressions “A or B”, “at least one of A or/and B”, or “one or more of A or/and B”, and the like may include any and all combinations of one or more of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

The terms, such as “first”, “second”, and the like used in this disclosure may be used to refer to various elements regardless of the order and/or the priority and to distinguish the relevant elements from other elements, but do not limit the elements. For example, “a first user device” and “a second user device” indicate different user devices regardless of the order or priority. For example, without departing the scope of the present disclosure, a first element may be referred to as a second element, and similarly, a second element may be referred to as a first element.

It will be understood that when an element (e.g., a first element) is referred to as being “(operatively or communicatively) coupled with/to” or “connected to” another element (e.g., a second element), it may be directly coupled with/to or connected to the other element or an intervening element (e.g., a third element) may be present. In contrast, when an element (e.g., a first element) is referred to as being “directly coupled with/to” or “directly connected to” another element (e.g., a second element), it should be understood that there are no intervening element (e.g., a third element).

According to the situation, the expression “configured to” used in this disclosure may be used as, for example, the expression “suitable for”, “having the capacity to”, “designed to”, “adapted to”, “made to”, or “capable of”. The term “configured to” must not mean only “specifically designed to” in hardware. Instead, the expression “a device configured to” may mean that the device is “capable of” operating together with another device or other components. For example, a “processor configured to (or set to) perform A, B, and C” may mean a dedicated processor (e.g., an embedded processor) for performing a corresponding operation or a generic-purpose processor (e.g., a central processing unit (CPU) or an application processor) which performs corresponding operations by executing one or more software programs which are stored in a memory device.

Terms used in this disclosure are used to describe specified embodiments and are not intended to limit the scope of the present invention. The terms of a singular form may include plural forms unless otherwise specified. All the terms used herein, which include technical or scientific terms, may have the same meaning that is generally understood by a person skilled in the art. It will be further understood that terms, which are defined in a dictionary and commonly used, should also be interpreted as is customary in the relevant related art and not in an idealized or overly formal unless expressly so defined in various embodiments of this disclosure. In some cases, even if terms are terms which are defined in this disclosure, they may not be interpreted to exclude embodiments of this disclosure.

An electronic device according to various embodiments of this disclosure may include at least one of, for example, smartphones, tablet personal computers (PCs), mobile phones, video telephones, electronic book readers, desktop PCs, laptop PCs, netbook computers, workstations, servers, personal digital assistants (PDAs), portable multimedia players (PMPs), Motion Picture Experts Group (MPEG-1 or MPEG-2) Audio Layer 3 (MP3) players, mobile medical devices, cameras, or wearable devices. According to various embodiments, the wearable device may include at least one of an accessory type (e.g., watches, rings, bracelets, anklets, necklaces, glasses, contact lens, or head-mounted-devices (HMDs), a fabric or garment-integrated type (e.g., an electronic apparel), a body-attached type (e.g., a skin pad or tattoos), or a bio-implantable type (e.g., an implantable circuit).

According to various embodiments, the electronic device may be a home appliance. The home appliances may include at least one of, for example, televisions (TVs), digital versatile disc (DVD) players, audios, refrigerators, air conditioners, cleaners, ovens, microwave ovens, washing machines, air cleaners, set-top boxes, home automation control panels, security control panels, TV boxes (e.g., Samsung HomeSync™, Apple TV™, or Google TV™), game consoles (e.g., Xbox™ or PlayStation™), electronic dictionaries, electronic keys, camcorders, electronic picture frames, and the like.

According to another embodiment, an electronic device may include at least one of various medical devices (e.g., various portable medical measurement devices (e.g., a blood glucose monitoring device, a heartbeat measuring device, a blood pressure measuring device, a body temperature measuring device, and the like), a magnetic resonance angiography (MRA), a magnetic resonance imaging (MRI), a computed tomography (CT), scanners, and ultrasonic devices), navigation devices, Global Navigation Satellite System (GNSS), event data recorders (EDRs), flight data recorders (FDRs), vehicle infotainment devices, electronic equipment for vessels (e.g., navigation systems and gyrocompasses), avionics, security devices, head units for vehicles, industrial or home robots, automatic teller's machines (ATMs), points of sales (POSs) of stores, or internet of things (e.g., light bulbs, various sensors, electric or gas meters, sprinkler devices, fire alarms, thermostats, street lamps, toasters, exercise equipment, hot water tanks, heaters, boilers, and the like).

According to an embodiment, the electronic device may include at least one of parts of furniture or buildings/structures, electronic boards, electronic signature receiving devices, projectors, or various measuring instruments (e.g., water meters, electricity meters, gas meters, or wave meters, and the like). According to various embodiments, the electronic device may be one of the above-described devices or a combination thereof. An electronic device according to an embodiment may be a flexible electronic device. Furthermore, an electronic device according to an embodiment of this disclosure may not be limited to the above-described electronic devices and may include other electronic devices and new electronic devices according to the development of technologies.

Hereinafter, electronic devices according to various embodiments will be described with reference to the accompanying drawings. In this disclosure, the term “user” may refer to a person who uses an electronic device or may refer to a device (e.g., an artificial intelligence electronic device) that uses the electronic device.

FIG. 1 illustrates an electronic device, according to various embodiments of this disclosure.

Referring to FIG. 1, according to various embodiments, an electronic device 101, 102, or 104, or a server 106 may be connected each other over a network 162 or a short range communication 164. The electronic device 101 may include a bus 110, a processor 120, a memory 130, an input/output interface 150, a display 160, and a communication interface 170. According to an embodiment, the electronic device 101 may not include at least one of the above-described elements or may further include other element(s).

For example, the bus 110 may interconnect the above-described elements 110 to 170 and may include a circuit for conveying communications (e.g., a control message and/or data) among the above-described elements.

The processor 120 may include one or more of a central processing unit (CPU), an application processor (AP), or a communication processor (CP). For example, the processor 120 may perform an arithmetic operation or data processing associated with control and/or communication of at least other elements of the electronic device 101.

The memory 130 may include a volatile and/or nonvolatile memory. For example, the memory 130 may store instructions or data associated with at least one other element(s) of the electronic device 101. According to an embodiment, the memory 130 may store software and/or a program 140. The program 140 may include, for example, a kernel 141, a middleware 143, an application programming interface (API) 145, and/or an application program (or “an application”) 147. At least a part of the kernel 141, the middleware 143, or the API 145 may be referred to as an “operating system (OS)”.

For example, the kernel 141 may control or manage system resources (e.g., the bus 110, the processor 120, the memory 130, and the like) that are used to execute operations or functions of other programs (e.g., the middleware 143, the API 145, and the application program 147). Furthermore, the kernel 141 may provide an interface that allows the middleware 143, the API 145, or the application program 147 to access discrete elements of the electronic device 101 so as to control or manage system resources.

The middleware 143 may perform, for example, a mediation role such that the API 145 or the application program 147 communicates with the kernel 141 to exchange data.

Furthermore, the middleware 143 may process task requests received from the application program 147 according to a priority. For example, the middleware 143 may assign the priority, which makes it possible to use a system resource (e.g., the bus 110, the processor 120, the memory 130, or the like) of the electronic device 101, to at least one of the application program 147. For example, the middleware 143 may process the one or more task requests according to the priority assigned to the at least one, which makes it possible to perform scheduling or load balancing on the one or more task requests.

The API 145 may be, for example, an interface through which the application program 147 controls a function provided by the kernel 141 or the middleware 143, and may include, for example, at least one interface or function (e.g., an instruction) for a file control, a window control, image processing, a character control, or the like.

The input/output interface 150 may play a role, for example, of an interface which transmits an instruction or data input from a user or another external device, to other element(s) of the electronic device 101. Furthermore, the input/output interface 150 may output an instruction or data, received from other element(s) of the electronic device 101, to a user or another external device.

The display 160 may include, for example, a liquid crystal display (LCD), a light-emitting diode (LED) display, an organic LED (OLED) display, a microelectromechanical systems (MEMS) display, or an electronic paper display. The display 160 may display, for example, various contents (e.g., a text, an image, a video, an icon, a symbol, and the like) to a user. The display 160 may include a touch screen and may receive, for example, a touch, gesture, proximity, or hovering input using an electronic pen or a part of a user's body.

For example, the communication interface 170 may establish communication between the electronic device 101 and an external device (e.g., the first external electronic device 102, the second external electronic device 104, or the server 106). For example, the communication interface 170 may be connected to the network 162 over wireless communication or wired communication to communicate with the external device (e.g., the external second electronic device 104 or the server 106).

The wireless communication may include cellular communication using at least one of, for example, long-term evolution (LTE), LTE Advanced (LTE-A), code division multiple access (CDMA), Wideband CDMA (WCDMA), universal mobile telecommunications system (UMTS), wireless Broadband (WiBro), global system for mobile communications (GSM), or the like as cellular communication protocol. According to an embodiment, the wireless communication may include at least one of, for example, wireless fidelity (Wi-Fi), Bluetooth, Bluetooth low energy (BLE), Zigbee, near field communication (NFC), magnetic secure transmission (MST), radio frequency (RF), body area network (BAN), or a global navigation satellite system (GNSS), or the like.

The MST may generate a pulse in response to transmission data using an electromagnetic signal, and the pulse may generate a magnetic field signal. The electronic device 101 may transfer the magnetic field signal to point of sale (POS), and the POS may detect the magnetic field signal using a MST reader. The POS may recover the data by converting the detected magnetic field signal to an electrical signal.

The GNSS may include at least one of, for example, a global positioning system (GPS), a global navigation satellite system (Glonass), a Beidou navigation satellite system (hereinafter referred to as “Beidou”), or an European global satellite-based navigation system (hereinafter referred to as “Galileo”) based on an available region, a bandwidth, or the like. Hereinafter, in this disclosure, “GPS” and “GNSS” may be interchangeably used.

The wired communication may include at least one of, for example, a universal serial bus (USB), a high definition multimedia interface (HDMI), a recommended standard-232 (RS-232), a plain old telephone service (POTS), or the like. The network 162 may include at least one of telecommunications networks, for example, a computer network (e.g., LAN or WAN), an Internet, or a telephone network.

Each of the first and second external electronic devices 102 and 104 may be a device of which the type is different from or the same as that of the electronic device 101. According to an embodiment, the server 106 may include a group of one or more servers. According to various embodiments, all or a portion of operations that the electronic device 101 will perform may be executed by another or plural electronic devices (e.g., the electronic device 102 or 104 or the server 106). According to an embodiment, in the case where the electronic device 101 executes any function or service automatically or in response to a request, the electronic device 101 may not perform the function or the service internally, but, alternatively additionally, it may request at least a portion of a function associated with the electronic device 101 from another device (e.g., the electronic device 102 or 104 or the server 106). The other electronic device (e.g., the electronic device 102 or 104 or the server 106) may execute the requested function or additional function and may transmit the execution result to the electronic device 101. The electronic device 101 may provide the requested function or service using the received result or may additionally process the received result to provide the requested function or service. To this end, for example, cloud computing, distributed computing, or client-server computing may be used.

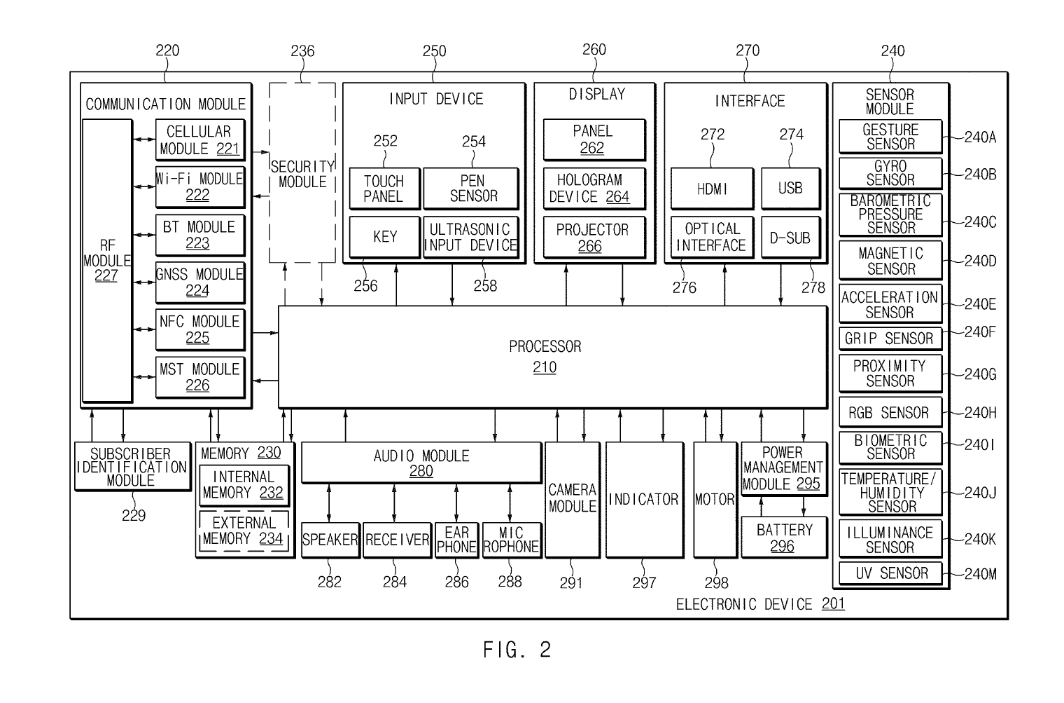

FIG. 2 illustrates a block diagram of an electronic device, according to various embodiments of the present disclosure.

Referring to FIG. 2, an electronic device 201 may include, for example, all or a part of the electronic device 101 illustrated in FIG. 1. The electronic device 201 may include one or more processors (e.g., an application processor (AP)) 210, a communication module 220, a subscriber identification module 229, a memory 230, a sensor module 240, an input device 250, a display 260, an interface 270, an audio module 280, a camera module 291, a power management module 295, a battery 296, an indicator 297, and a motor 298.

The processor 210 may drive, for example, an operating system (OS) or an application to control a plurality of hardware or software elements connected to the processor 210 and may process and compute a variety of data. For example, the processor 210 may be implemented with a System on Chip (SoC). According to an embodiment, the processor 210 may further include a graphic processing unit (GPU) and/or an image signal processor. The processor 210 may include at least a part (e.g., a cellular module 221) of elements illustrated in FIG. 2. The processor 210 may load an instruction or data, which is received from at least one of other elements (e.g., a nonvolatile memory), into a volatile memory and process the loaded instruction or data. The processor 210 may store a variety of data in the nonvolatile memory.

The communication module 220 may be configured the same as or similar to the communication interface 170 of FIG. 1. The communication module 220 may include the cellular module 221, a Wi-Fi module 222, a Bluetooth (BT) module 223, a GNSS module 224 (e.g., a GPS module, a Glonass module, a Beidou module, or a Galileo module), a near field communication (NFC) module 225, a MST module 226 and a radio frequency (RF) module 227.

The cellular module 221 may provide, for example, voice communication, video communication, a character service, an Internet service, or the like over a communication network. According to an embodiment, the cellular module 221 may perform discrimination and authentication of the electronic device 201 within a communication network by using the subscriber identification module (e.g., a SIM card) 229. According to an embodiment, the cellular module 221 may perform at least a portion of functions that the processor 210 provides. According to an embodiment, the cellular module 221 may include a communication processor (CP).

Each of the Wi-Fi module 222, the BT module 223, the GNSS module 224, the NFC module 225, or the MST module 226 may include a processor for processing data exchanged through a corresponding module, for example. According to an embodiment, at least a part (e.g., two or more) of the cellular module 221, the Wi-Fi module 222, the BT module 223, the GNSS module 224, the NFC module 225, or the MST module 226 may be included within one Integrated Circuit (IC) or an IC package.

For example, the RF module 227 may transmit and receive a communication signal (e.g., an RF signal). For example, the RF module 227 may include a transceiver, a power amplifier module (PAM), a frequency filter, a low noise amplifier (LNA), an antenna, or the like. According to another embodiment, at least one of the cellular module 221, the Wi-Fi module 222, the BT module 223, the GNSS module 224, the NFC module 225, or the MST module 226 may transmit and receive an RF signal through a separate RF module.

The subscriber identification module 229 may include, for example, a card and/or embedded SIM that includes a subscriber identification module and may include unique identify information (e.g., integrated circuit card identifier (ICCID)) or subscriber information (e.g., integrated mobile subscriber identity (IMSI)).

The memory 230 (e.g., the memory 130) may include an internal memory 232 or an external memory 234. For example, the internal memory 232 may include at least one of a volatile memory (e.g., a dynamic random access memory (DRAM), a static RAM (SRAM), a synchronous DRAM (SDRAM), or the like), a nonvolatile memory (e.g., a one-time programmable read only memory (OTPROM), a programmable ROM (PROM), an erasable and programmable ROM (EPROM), an electrically erasable and programmable ROM (EEPROM), a mask ROM, a flash ROM, a flash memory (e.g., a NAND flash memory or a NOR flash memory), or the like), a hard drive, or a solid state drive (SSD).

The external memory 234 may further include a flash drive such as compact flash (CF), secure digital (SD), micro secure digital (Micro-SD), mini secure digital (Mini-SD), extreme digital (xD), a multimedia card (MMC), a memory stick, or the like. The external memory 234 may be operatively and/or physically connected to the electronic device 201 through various interfaces.

A security module 236 may be a module that includes a storage space of which a security level is higher than that of the memory 230 and may be a circuit that guarantees safe data storage and a protected execution environment. The security module 236 may be implemented with a separate circuit and may include a separate processor. For example, the security module 236 may be in a smart chip or a secure digital (SD) card, which is removable, or may include an embedded secure element (eSE) embedded in a fixed chip of the electronic device 201. Furthermore, the security module 236 may operate based on an operating system (OS) that is different from the OS of the electronic device 201. For example, the security module 236 may operate based on java card open platform (JCOP) OS.

The sensor module 240 may measure, for example, a physical quantity or may detect an operation state of the electronic device 201. The sensor module 240 may convert the measured or detected information to an electric signal. For example, the sensor module 240 may include at least one of a gesture sensor 240A, a gyro sensor 240B, a barometric pressure sensor 240C, a magnetic sensor 240D, an acceleration sensor 240E, a grip sensor 240F, the proximity sensor 240G, a color sensor 240H (e.g., red, green, blue (RGB) sensor), a biometric sensor 240I, a temperature/humidity sensor 240J, an illuminance sensor 240K, or an UV sensor 240M. Although not illustrated, additionally or generally, the sensor module 240 may further include, for example, an E-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit for controlling at least one or more sensors included therein. According to an embodiment, the electronic device 201 may further include a processor that is a part of the processor 210 or independent of the processor 210 and is configured to control the sensor module 240. The processor may control the sensor module 240 while the processor 210 remains at a sleep state.

The input device 250 may include, for example, a touch panel 252, a (digital) pen sensor 254, a key 256, or an ultrasonic input unit 258. For example, the touch panel 252 may use at least one of capacitive, resistive, infrared and ultrasonic detecting methods. Also, the touch panel 252 may further include a control circuit. The touch panel 252 may further include a tactile layer to provide a tactile reaction to a user.

The (digital) pen sensor 254 may be, for example, a part of a touch panel or may include an additional sheet for recognition. The key 256 may include, for example, a physical button, an optical key, a keypad, or the like. The ultrasonic input device 258 may detect (or sense) an ultrasonic signal, which is generated from an input device, through a microphone (e.g., a microphone 288) and may check data corresponding to the detected ultrasonic signal.

The display 260 (e.g., the display 160) may include a panel 262, a hologram device 264, or a projector 266. The panel 262 may be the same as or similar to the display 160 illustrated in FIG. 1. The panel 262 may be implemented, for example, to be flexible, transparent or wearable. The panel 262 and the touch panel 252 may be integrated into a single module. The hologram device 264 may display a stereoscopic image in a space using a light interference phenomenon. The projector 266 may project light onto a screen so as to display an image. For example, the screen may be arranged in the inside or the outside of the electronic device 201. According to an embodiment, the panel 262 may include a pressure sensor (or a force sensor) capable of measuring the intensity of pressure associated with the touch of a user. The pressure sensor may be integrally implemented with the touch panel 252 or may be implemented with one or more sensors independently of the touch panel 252. According to an embodiment, the display 260 may further include a control circuit for controlling the panel 262, the hologram device 264, or the projector 266.

The interface 270 may include, for example, a high-definition multimedia interface (HDMI) 272, a universal serial bus (USB) 274, an optical interface 276, or a D-subminiature (D-sub) 278. The interface 270 may be included, for example, in the communication interface 170 illustrated in FIG. 1. Additionally or generally, the interface 270 may include, for example, a mobile high definition link (MHL) interface, a SD card/multi-media card (MMC) interface, or an infrared data association (IrDA) standard interface.

The audio module 280 may convert a sound and an electric signal in dual directions. At least a part of the audio module 280 may be included, for example, in the input/output interface 150 illustrated in FIG. 1. The audio module 280 may process, for example, sound information that is input or output through a speaker 282, a receiver 284, an earphone 286, or the microphone 288.

For example, the camera module 291 may shoot a still image or a video. According to an embodiment, the camera module 291 may include at least one or more image sensors (e.g., a front sensor or a rear sensor), a lens, an image signal processor (ISP), or a flash (e.g., an LED or a xenon lamp).

The power management module 295 may manage, for example, power of the electronic device 201. According to an embodiment, a power management integrated circuit (PMIC), a charger IC, or a battery or fuel gauge may be included in the power management module 295. The PMIC may have a wired charging method and/or a wireless charging method. The wireless charging method may include, for example, a magnetic resonance method, a magnetic induction method or an electromagnetic method and may further include an additional circuit, for example, a coil loop, a resonant circuit, or a rectifier, and the like. The battery gauge may measure, for example, a remaining capacity of the battery 296 and a voltage, current or temperature thereof while the battery is charged. The battery 296 may include, for example, a rechargeable battery and/or a solar battery.

The indicator 297 may display a specific state of the electronic device 201 or a part thereof (e.g., the processor 210), such as a booting state, a message state, a charging state, and the like. The motor 298 may convert an electrical signal into a mechanical vibration and may generate the following effects: vibration, haptic, and the like. Although not illustrated, a processing device (e.g., a GPU) for supporting a mobile TV may be included in the electronic device 201. The processing device for supporting the mobile TV may process media data according to the standards of digital multimedia broadcasting (DMB), digital video broadcasting (DVB), MediaFlo™, or the like.

Each of the above-mentioned elements of the electronic device according to various embodiments of the present disclosure may be configured with one or more components, and the names of the elements may be changed according to the type of the electronic device. In various embodiments, the electronic device may include at least one of the above-mentioned elements, and some elements may be omitted or other additional elements may be added. Furthermore, some of the elements of the electronic device according to various embodiments may be combined with each other so as to form one entity, so that the functions of the elements may be performed in the same manner as before the combination.

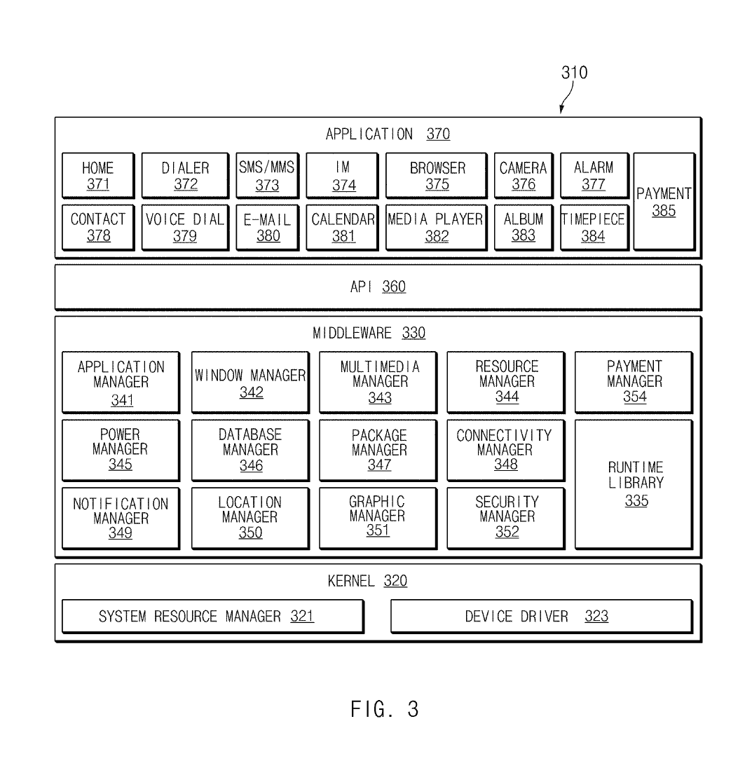

FIG. 3 illustrates a block diagram of a program module, according to various embodiments of this disclosure.

According to at least one embodiment, a program module 310 (for example, the program 140 of FIG. 1) may include an operating system (OS) to control resources associated with an electronic device (e.g., the electronic device 101), and/or diverse applications (e.g., the application program 147) driven on the OS. The OS may be, for example, Android, iOS, Windows, Symbian, Tizen, or Bada.

The program module 310 may include a kernel 320, a middleware 330, an application programming interface (API) 360, and/or an application 370. At least a portion of the program module 310 may be preloaded on an electronic device or may be downloadable from an external electronic device (e.g., the electronic device 102 or 104, the server 106, or the like).

The kernel 320 (e.g., the kernel 141) may include, for example, a system resource manager 321 or a device driver 323. The system resource manager 321 may perform control, allocation, or retrieval of system resources. According to an embodiment, the system resource manager 321 may include a process managing unit, a memory managing unit, or a file system managing unit. The device driver 323 may include, for example, a display driver, a camera driver, a Bluetooth driver, a shared memory driver, a USB driver, a keypad driver, a Wi-Fi driver, an audio driver, or an inter-process communication (IPC) driver.

The middleware 330 may provide, for example, a function that the application 370 needs in common, or may provide diverse functions to the application 370 through the API 360 to allow the application 370 to efficiently use limited system resources of the electronic device. According to an embodiment, the middleware 330 (e.g., the middleware 143) may include at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, a security manager 352, or a payment manager 354.

The runtime library 335 may include, for example, a library module that is used by a compiler to add a new function through a programming language while the application 370 is being executed. The runtime library 335 may perform input/output management, memory management, or capacities about arithmetic functions.

The application manager 341 may manage, for example, a life cycle of at least one application of the application 370. The window manager 342 may manage a graphic user interface (GUI) resource that is used in a screen. The multimedia manager 343 may identify a format necessary for playing diverse media files, and may perform encoding or decoding of media files by using a codec suitable for the format. The resource manager 344 may manage resources such as a storage space, memory, or source code of at least one application of the application 370.

The power manager 345 may operate, for example, with a basic input/output system (BIOS) to manage a battery or power, and may provide power information for an operation of an electronic device. The database manager 346 may generate, search for, or modify database that is to be used in at least one application of the application 370. The package manager 347 may install or update an application that is distributed in the form of package file.

The connectivity manager 348 may manage, for example, wireless connection such as Wi-Fi or Bluetooth. The notification manager 349 may display or notify an event such as arrival message, appointment, or proximity notification in a mode that does not disturb a user. The location manager 350 may manage location information about an electronic device. The graphic manager 351 may manage a graphic effect that is provided to a user, or manage a user interface relevant thereto. The security manager 352 may provide a general security function necessary for system security, user authentication, or the like. According to an embodiment, in the case where an electronic device (e.g., the electronic device 101) includes a telephony function, the middleware 330 may further include a telephony manager for managing a voice or video call function of the electronic device.

The middleware 330 may include a middleware module that combines diverse functions of the above-described elements. The middleware 330 may provide a module specialized to each OS kind to provide differentiated functions. Additionally, the middleware 330 may dynamically remove a part of the preexisting elements or may add new elements thereto.

The API 360 (e.g., the API 145) may be, for example, a set of programming functions and may be provided with a configuration that is variable depending on an OS. For example, in the non-limiting case where an OS is Android or iOS, it may provide one API set per platform. In the case where an OS is the Tizen, it may provide two or more API sets per platform.

The application 370 (e.g., the application program 147) may include, for example, one or more applications capable of providing functions for a home 371, a dialer 372, an SMS/MMS 373, an instant message (IM) 374, a browser 375, a camera 376, an alarm 377, a contact 378, a voice dial 379, an e-mail 380, a calendar 381, a media player 382, an album 383, a timepiece 384 and a payment 385 or for offering health care (e.g., measuring an exercise quantity, blood sugar, or the like) or environment information (e.g., information of barometric pressure, humidity, temperature, or the like).

According to an embodiment, the application 370 may include an application (hereinafter referred to as “information exchanging application” for descriptive convenience) to support information exchange between an electronic device (e.g., the electronic device 101) and an external electronic device (e.g., the electronic device 102 or 104). The information exchanging application may include, for example, a notification relay application for transmitting specific information to an external electronic device, or a device management application for managing the external electronic device.

For example, the notification relay application may include a function of transmitting notification information, which arise from other applications (e.g., applications for SMS/MMS, e-mail, health care, or environmental information), to an external electronic device (e.g., the electronic device 102 or 104). Additionally, the information exchanging application may receive, for example, notification information from an external electronic device and provide the notification information to a user.

The device management application may manage (e.g., install, delete, or update), for example, at least one function (e.g., turn-on/turn-off of an external electronic device itself (or a part of elements) or adjustment of brightness (or resolution) of a display) of the external electronic device which communicates with the electronic device, an application running in the external electronic device, or a service (e.g., a call service, a message service, or the like) provided from the external electronic device.

According to an embodiment, the application 370 may include an application (e.g., a health care application of a mobile medical device) that is assigned in accordance with an attribute of an external electronic device (e.g., the electronic device 102 or 104). According to an embodiment, the application 370 may include an application that is received from an external electronic device (e.g., the server 106 or the electronic device 102 or 104). According to an embodiment, the application 370 may include a preloaded application or a third party application that is downloadable from a server. The names of elements of the program module 310 according to the embodiment may be modifiable depending on kinds of operating systems.

According to various embodiments, at least a portion of the program module 310 may be implemented by software, firmware, hardware, or a combination of two or more thereof. At least a portion of the program module 310 may be implemented (e.g., executed), for example, by the processor (e.g., the processor 210). At least a portion of the program module 310 may include, for example, modules, programs, routines, sets of instructions, processes, or the like for performing one or more functions.

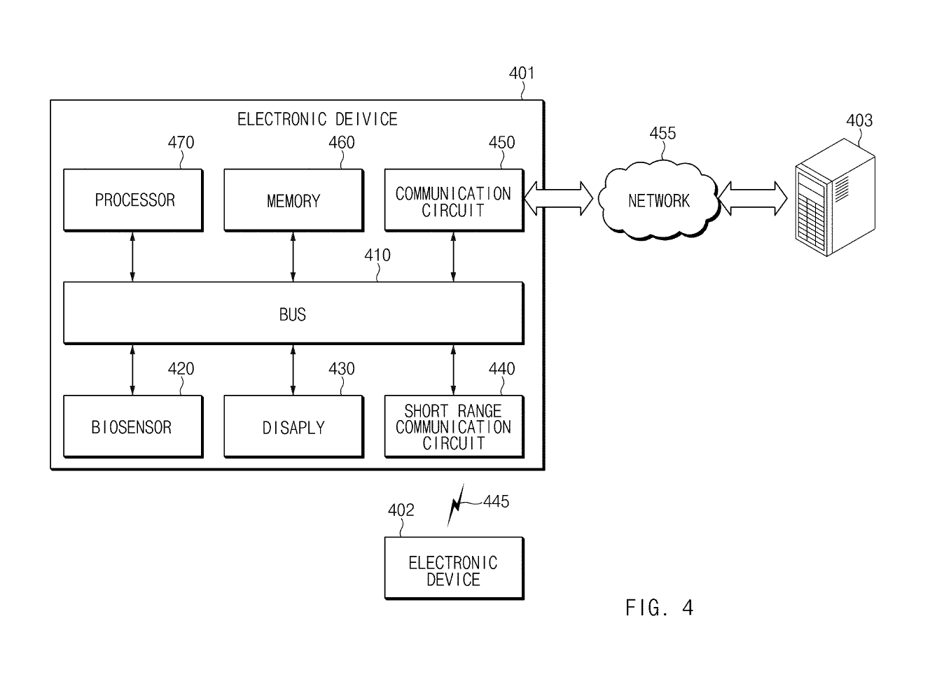

FIG. 4 illustrates, a block diagram of an electronic device according to certain embodiments of this disclosure.

Referring to FIG. 4, an electronic device 401 according to at least one embodiment may include, for example, a bus 410, a biosensor 420, a display 430, a short range communication circuit 440, a communication circuit 450, a memory 460, and/or a processor 470. According to various embodiments, the electronic device 401 may be implemented without some of the elements illustrated in FIG. 4 or may be implemented to further include one or more elements not illustrated in FIG. 4.

For example, the bus 410 according to some embodiments of the present disclosure may electrically interconnect the elements 420 to 470 of the electronic device 401. The bus 410 may include a circuit that transfers a communication message (e.g., a control message and/or data) between the elements.

The biosensor 420 according to certain embodiments of the present disclosure may obtain values of various health parameters that are originated from a user's body or measured from the user's activity. The biosensor 420 may convert the values of the health parameters into digital values and may provide the converted digital values to the processor 470. According to various embodiments, the biosensor 420 may include a driver IC or may be connected to a separate low-power control circuit (e.g., a micro controller unit (MCU), so-called a sensor hub).

The biosensor 420 according to various embodiments of the present disclosure may include various sensors that obtain the values of health parameters such as a step count, a heart rate, an electrocardiogram (ECG), an iris, a blood pressure, glucose, a weight, a body fat, oxygen saturation, a body temperature, and skin conductivity. In addition, to obtain the values of health parameters, various types of sensor modules 240A to 240M illustrated in FIG. 2 may be utilized in various embodiments of the present disclosure.

According to at least one embodiment of the present disclosure, the display 430 may display various contents (e.g., a text, an image, a video, an icon, an object, a symbol, and the like) under control of the processor 470. The display 430 may include a touchscreen and may receive, for example, a touch, gesture, proximity, or hovering input using an electronic pen or a part of the user's body. The display 430 may output execution screens of various types of software, a graphic user interface (GUI), or the like.

The short range communication circuit 440 according to certain embodiments of the present disclosure may establish a connection with an external electronic device 402. For example, the connection with the external electronic device 402 may include device to device (D2D) connection via a short range communication protocol, such as Bluetooth, Bluetooth low energy (BLE), or Zigbee.

The communication circuit 450 according to certain embodiments of the present disclosure may access a network 455 by wire or wirelessly to establish communication with a server 403. The communication circuit 450 may connect with the network 455 by using at least one of, for example, LTE, LTE-A, CDMA, WCDMA, UMTS, WiBro, or GSM as a cellular communication protocol. The network 455 may include a computer network such as LAN or WAN.

According to various embodiments of the present disclosure, the memory 460 may store an instruction, information, or data associated with the operations of the elements 420 to 470 included in the electronic device 401. For example, the memory 460 may store various types of applications (e.g., a web browser application, an image viewer application, a messenger application in which a chatbot is embedded, a speech recognition application, or a map application), and data (e.g., a health profile, a goal profile, or the like) necessary for operations of the applications. According to at least one embodiment, a software developer's kit (SDK) for executing an instruction corresponding to a health content providing method according to various embodiments of the present disclosure may be installed in the various types of applications.

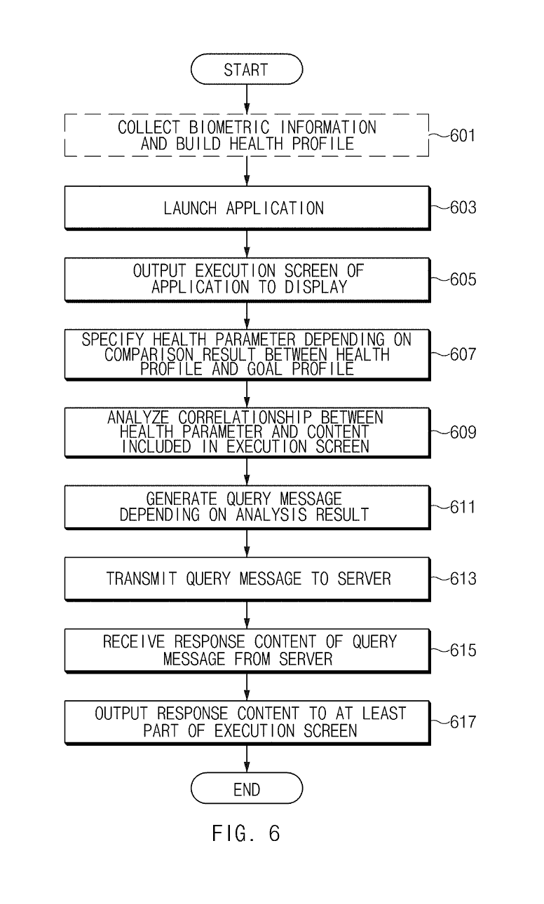

According to some embodiments, the memory 460 may store instructions that, when executed, cause the processor 470 to perform a health content providing method (for example, the method shown in the non-limiting example of FIG. 6 herein) disclosed in this specification. After the instructions are implemented with, for example, software such as an application program, an operating system (OS), or firmware, the instructions may be stored in the memory 460 or may be embedded in hardware.

For example, the processor 470 according to an embodiment of the present disclosure may be electrically connected to the elements 420 to 460 included in the electronic device 401, through the bus 410 and may execute operations or data processing associated with control and/or communication of the elements 420 to 460 included in the electronic device 401.

According to certain embodiments, the processor 470 may execute or launch various applications (e.g., a web browser application and the like). The processor 470 may output an execution screen including specific content (a screen according to specific activity of an application) to the display 430 in response to the launching of an application.

According to at least one embodiment, the processor 470 may analyze correlations between a specified health parameter and the content included in the execution screen. The specified health parameter may include various parameters that are originated from the user's body or are measured from the user's activity. For example, the specified health parameter may include at least one of a step count, a heart rate, an ECG, a blood pressure, glucose, a weight, a body fat, oxygen saturation, a body temperature, or skin conductivity. The health parameters listed above are illustrative, and there may be a lot of other health parameters. For example, the health parameter may include a cholesterol index, various types of allergies, obesity, a state of a body part (e.g., eyes or facial skin), or the like.

The specified health parameter according to certain embodiments of the present disclosure may be specified in a variety of ways. According to at least oen embodiment, the memory 460 may store a health profile including a first group of heath parameters, and a goal profile including a second group of heath parameters being a goal value of the first group of heath parameters. Biometric information corresponding to the first group of heath parameters of the health profile may be collected from the biosensor 420 or from the external electronic device 402 through the short range communication circuit 440. By way of non-limiting example, the health profile and the goal profile as illustrated in Table 1 below may be stored in the memory 460.

TABLE 1

| | Health profile | Target profile |
|---|---|---|
| Heath | (a first group of | (a second group of |
| parameter | heath parameters) | heath parameters) |
| Weight | — | 70 kg |
| Body fat rate | 21.2% | — |
| Glucose | 210 mg/dl | 70-110 mg/dl |
| Blood pressure | 98 mmHg | 80-120 mmHg |
| Step count | 11847 steps | 10000 steps |
| . . . | . . . | . . . |

The processor 470 according to certain embodiments of the present disclosure may specify the specified health parameter depending on the comparison result between the first group of heath parameters included in the health profile and the second group of heath parameters included in the goal profile. According to an embodiment, the processor 470 may extract a common health parameter from the first group of heath parameters and the second group of heath parameters; if a difference between a value of the common health parameter included in the health profile and a value of the common health parameter included in the goal profile is out of a specified range, the processor 470 may specify the common health parameter as the specified health parameter.

For example, as illustrated in Table 1, the processor 470 may extract the common health parameter from the first group of heath parameters and the second group of heath parameters. Since there is no item “weight” in the health profile and there is no item “body fat rate” in the goal profile, “glucose”, “blood pressure”, and “step count” may be extracted as common health parameters. The processor 470 may determine whether a difference between values of “glucose”, “blood pressure”, and “step count” is out of the specified range. In Table 1, the “glucose” value of the health profile is 210 mg/dl, and is twice as high as the maximum value (110 mg/dl) of a glucose range set in the goal profile. In addition, the “blood pressure” value of the health profile is 98 mmHg, and is within the blood pressure range (80 to 120 mmHg) set in the goal profile. Meanwhile, the step count is 11847 steps, and already exceeds the goal step count set in the goal profile. Accordingly, the processor 470 may specify “glucose” of health parameters included in the health profile and the goal profile, as the specified health parameter. The processor 470 may analyze correlations between the specified “glucose” as described above and content included in an application execution screen.

According to certain embodiments, the processor 470 may specify the specified health parameter based on the analysis result of content (e.g., an image, a text, or the like) output to the display 430. For example, the processor 470 may analyze the content output to the display 430, may extract a health parameter, which is out of a normal range or a goal range, and may specify the health parameter as the specified health parameter. That is, the processor 470 may statically specify the specified health parameter based on the comparison between the health profile and the goal profile or may dynamically specify the specified health parameter based on the analysis result of the content output to the display 430.

According to at least one embodiment, the processor 470 may generate a specified query message depending on the analysis result of a correlation between the specified health parameter and the content included in the execution screen and may transmit the specified query message to the server 403 through the communication circuit 450. Afterwards, the processor 470 may receive the response content of the query message from the server 403 and may output the corresponding response content to at least part of an application execution screen.

According to certain embodiments, the response content may include information associated with the specified health parameter (e.g., “glucose”). Specifically, the information associated with the specified health parameter may include medical information about the specified health parameter, and, for another example, may include nutrition information, exercise information, or the like for improving the specified health parameter.

The above-described operation of the processor 470 is provided as one non-limiting example. Embodiments may not be limited thereto. Also, in this specification, at least some of operations described as operations of an “electronic device” should be understood as operations of the processor 470.

The external electronic device 402 according to certain embodiments of the present disclosure may correspond to a device the same as or similar to the electronic device 401. According to some embodiments, the external electronic device 402 may include a biosensor, and may provide biometric information obtained from the corresponding biosensor to the electronic device 401 through short range communication. For example, the external electronic device 402 may include at least one of an accessory type (e.g., watches, rings, bracelets, anklets, necklaces, glasses, contact lens, or head-mounted devices (HMDs), a fabric or garment-integrated type (e.g., an electronic apparel), a body-attached type (e.g., a skin pad or tattoos), or an implantable type.

The server 403 according to certain embodiments of the present disclosure may provide the electronic device 401 with health content as a response to the query message received from the electronic device 401. In some embodiments of the present disclosure, the health content may be referred to “response content”. The server 403 may include a health content database necessary to provide the health content, and an artificial intelligence platform for constituting the health content.

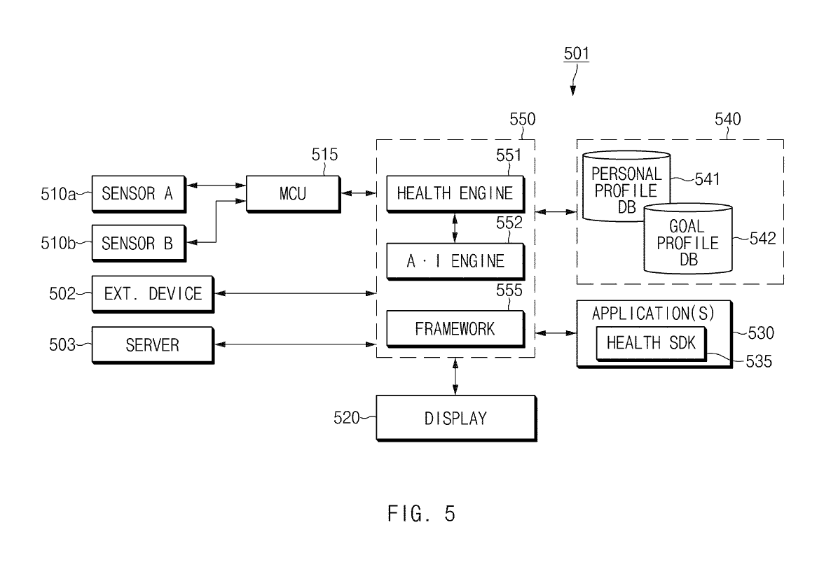

FIG. 5 illustrates a block diagram of an electronic device according to embodiments of the present disclosure.

Referring to the non-limiting example of FIG. 5, an electronic device 501 according to an embodiment is illustrated. The electronic device 501 illustrated in FIG. 5 may include biosensors 510a and 510b, a MCU 515, a display 520, an application program 530, a database 540, and a processor 550. The electronic device 501 illustrated in FIG. 5 may correspond to viewing the electronic device 401 illustrated in FIG. 4.

According to certain embodiments, the biosensors 510a and 510b may obtain biometric information corresponding to the pre-allocated health parameter, under control of the MCU 515 being a low-power processor. The obtained biometric information may be provided to a health engine 551 through the MCU 515 after being converted into a digital value.

According to at least one embodiment, the biometric information corresponding to the health parameter may be received from an external electronic device 502 through short range communication. The external device may correspond to the external electronic device 402 illustrated in FIG. 4.

Under control of the processor 550, the display 520 may output the execution screen of the application program 530 or may output health content (response content) received from a server 503.

The application program 530 according to certain embodiments of the present disclosure may include various application programs such as a web browser application, an image viewer application, a messenger application in which a chatbot is embedded, a speech recognition application, a map application, or the like. A SDK for executing an instruction corresponding to a health content providing method according to various embodiments of the present disclosure may be installed in the application program 530.

As a non-limiting example, the database 540 may be built on the memory of the electronic device 501. The database 540 may include a user's personal health profile DB 541 or goal profile DB 542 corresponding to the goal value of the health profile.

The health profile DB 541 may comprise a first group of heath parameters, and the goal profile DB 542 may comprise a second group of heath parameters being the goal value of the first group of heath parameters.

According to certain embodiments, the health profile DB 541 may be updated in real time based on biometric information obtained from the biosensors 510a and 510b and/or biometric information obtained from the external electronic device 502. That is, various health parameters may be updated to indicate the current health state or current activity state of the user. According to various embodiments, the health profile DB 541 may be updated based on the user's health information obtained from a fitness center that the user joins. According to some embodiments, the health profile DB 541 of the user may be shared with electronic devices of peers or an electronic device of the user's doctor.

According to at least one embodiment, the goal profile DB 542 may be variously set depending on user intent. According to some embodiments, the goal profile DB 542 may be set to indicate the health profile of a celebrity. That is, the goal profile DB 542 may comprise a health profile that the user targets. According to some embodiments, the health profile of the celebrity may be distributed in a form capable of being commercially traded.

According to some embodiments of the present disclosure, the health engine 551, an AI engine 552, and a framework 555 may correspond to a software module driven by a computing resource of the processor 550. Accordingly, in some embodiments, the function and the operation performed by the health engine 551, the AI engine 552, and/or the framework 555 are the function and the operation of the processor 550.

According to certain embodiments, the health engine 551 may manage the health profile DB 541 of the user, based on the biometric information obtained from the biosensors 510a and 510b and/or the external electronic device 502. As such, the health profile DB 541 may be updated to indicate the current health state and/or current activity state of the user.

According to at least one embodiment, the health engine 551 may specify a specific health parameter, based on the comparison result between the health profile DB 541 and the goal profile DB 542 of the user. The health parameter specified by the health engine 551 may correspond to a parameter, which affects the health of the user, as a health parameter that is out of an ordinary allowable range. The health parameter specified by the health engine 551 may be provided to the AI engine 552.

According to an embodiment, the AI engine 552 may analyze correlations between the health parameter specified by the health engine 551 and the content included in the execution screen of the application 530 and may generate a specified query message depending on the analysis result. The AI engine 552 may utilize optical character recognition (OCR), image object recognition, or the like for the purpose of recognizing the content (a text, an image, or the like) included in the execution screen. For example, in the case where the health parameter specified by the health engine 551 is “glucose”, and an image of a high-sugar beverage is displayed on the execution screen of a web browser application, the AI engine 552 may recognize the high-sugar beverage and may analyze correlations between “glucose” and the “high-sugar beverage”, and then may generate a query message as a result. For another example, if receiving a text (e.g., a text entered in a search field) from the user, the AI engine 552 may analyze correlations between the health parameter and the entered text. The AI engine 552 may generate the query message that is based on the entered text, depending on the analysis result.

The AI engine 552 according to some embodiments of the present disclosure may transmit the specified query message to the server 503 and may receive health content (response content) in response to the query message. As a non-limiting example, the health content may include information associated with “glucose”. In more detail, the health content may include medical information about how “high-sugar beverage” affect “high blood sugar”, for another example, may include information about a recommended food for improving high “glucose”, information about a recommended exercise for lowering “glucose”, or the like. This health content may be output on the display 520 through the framework 555.

FIG. 6 illustrates a flowchart of a health content providing method, according to certain embodiments of this disclosure.

Referring to the non-limiting example shown in FIG. 6, the health content providing method according to some embodiments may include operations 601 to 617. For example, operations 601 to 617 may be performed by the electronic device 401 illustrated in FIG. 4 or the electronic device 501 illustrated in FIG. 5. Operations 601 to 617 may be respectively implemented with, for example, instructions capable of being performed (or executed) by the processor 470 of the electronic device 401. The instructions may be stored in, for example, a computer-readable recording medium or the memory 460 illustrated in FIG. 4. Hereinafter, aspects of operations 601 617 may be described in this non-limiting example with reference to elements shown in FIG. 4 herein.

According to certain embodiments, at operation 601, the processor 470 of the electronic device 401 may collect biometric information corresponding to a health parameter and may build the personal health profile of a user in the memory 460, based on the biometric information. The biometric information may be collected from the embedded biosensor 420 or the external electronic device 402. For example, the specified health parameter may include, but not limited to, a step count, a heart rate, an ECG, a blood pressure, glucose, a weight, a body fat, oxygen saturation, a body temperature, skin conductivity, or the like.

According to some embodiments, at operation 603, the processor 470 may launch a specified application. The specified application may include at least one of a web browser application, an image viewer application, a messenger application in which a chatbot is embedded, a speech recognition application, or a map application.

According to some embodiments, at operation 605, the processor 470 may output the execution screen of a specified application on the display 430.

In the non-limiting example of FIG. 6, at operation 607, the processor 470 may specify a specific health parameter depending on the comparison result between a first group of heath parameters included in a health profile and a second group of heath parameters included in a goal profile. For example, the processor 470 may extract a common health parameter from the first group of heath parameters included in the health profile and the second group of heath parameters included in the goal profile; if a difference between a value of the common health parameter included in the health profile and a value of the common health parameter included in the goal profile is out of a specified range, the processor 470 may specify the common health parameter (in more detail, refer to Table 1).

According to certain embodiments, at operation 609, the processor 470 may analyze correlations between the health parameter specified in operation 607 and the content included in an execution screen output in operation 605. For example, the processor 470 may analyze whether a parameter that affects the health parameter specified in operation 607 is included in the content included in the execution screen.

According to certain embodiments, at operation 611, the processor 470 may generate a specified query message depending on the result analyzed in operation 609.

According to certain embodiments, at operation 613, the processor 470 may transmit the query message generated in operation 611, to the server 403 through the network 455.

According to certain embodiments, at operation 615, the processor 470 may receive the response content (i.e., health content) of the query message from the server 403. The response content may include information (recommended medical information, recommended nutrition information, recommended exercise information, or the like) associated with the health parameter specified in operation 607.

According to certain embodiments, at operation 617, the processor 470 may output the response content received in operation 615, to at least part of the execution screen.

According to embodiments of a health content providing method and an electronic device performing the same as described above, optimal health content is provided to the user based on the current health state and/or the current activity state of the user. In particular, the health content that is optimized to use the electronic device of the user may be provided by interworking with various application programs.

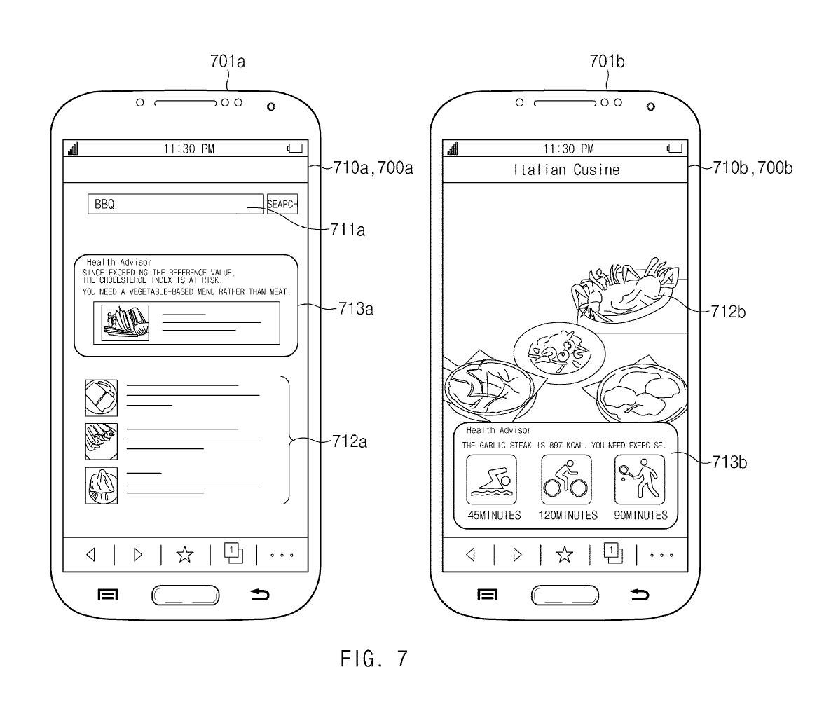

FIG. 7 illustrates a view of aspects of a web browser application according to embodiments of this disclosure.

In the non-limiting example of FIG. 7, a user may execute a web browser application by using an electronic device 701a and may access a search engine website. For example, the search engine website may be output to a display 710a of the electronic device 701a depending on the execution of the web browser application. Afterwards, the user may enter “BBQ” as a search keyword in a search field 711a of the search engine website. The electronic device 701a may receive the search result of “BBQ” from the search engine through a specific interaction (e.g., the selection of a search button). Meanwhile, a health parameter may be pre-specified as a “cholesterol index”.

According to certain embodiments, the search field 711a in which “BBQ” is described, content 712a in which the search result of “BBQ” is included, and/or health content 713a provided from a server may be included in an execution screen 700a of the web browser application. For the purpose of displaying the health content 713a on the execution screen 700a, for example, the electronic device 701a may perform the following operation.

According to at least one embodiment, the electronic device 701a may analyze correlations between the “cholesterol index” being the pre-specified health parameter and the content 712a in which the search result of “BBQ” is included, and may generate a specific query message depending on the analysis result. The electronic device 701a may transmit the query message to the server and may receive the health content 713a from the server in response to the query message.

As illustrated in FIG. 7, the health content 713a may include a warning about the “cholesterol index” and information about a recommended food. The health content 713a may be output to a part of the execution screen 700a of the web browser application. As such, when receiving the search result of “BBQ”, the user may receive health content associated with the “cholesterol index” together.

As a further example, the user may execute the web browser application by using an electronic device 701b and may access a website of restaurant “Italian Cuisine”. A website screen 700b of the restaurant may be output to a display 710b of the electronic device 701b by the access. Meanwhile, a health parameter may be pre-specified as “calorie”.

According to some embodiments, foods 712b provided by the restaurant and health content 713b provided from the server may be included in the execution screen 700b of the web browser application. For the purpose of displaying the health content 713b on the execution screen 700b, for example, the electronic device 701b may perform the following operation.

According to some embodiments, the electronic device 701b may analyze correlations between “calories” as the pre-specified health parameter and the foods 712b provided by the restaurant, and may generate a specific query message depending on the analysis result. The electronic device 701b may transmit the query message to the server and may receive the health content 713b from the server in response to the query message.

As illustrated in the non-limiting example of FIG. 7, the health content 713b may include the “calorie” of the foods 712b displayed on the execution screen 700b and information about exercise quantity necessary to consume the corresponding calorie. The health content 713b may be output to a part of the execution screen 700b of the web browser application. As such, when reading the website of a restaurant, the user may receive health content associated with the “calorie” of foods provided by the corresponding restaurant together.

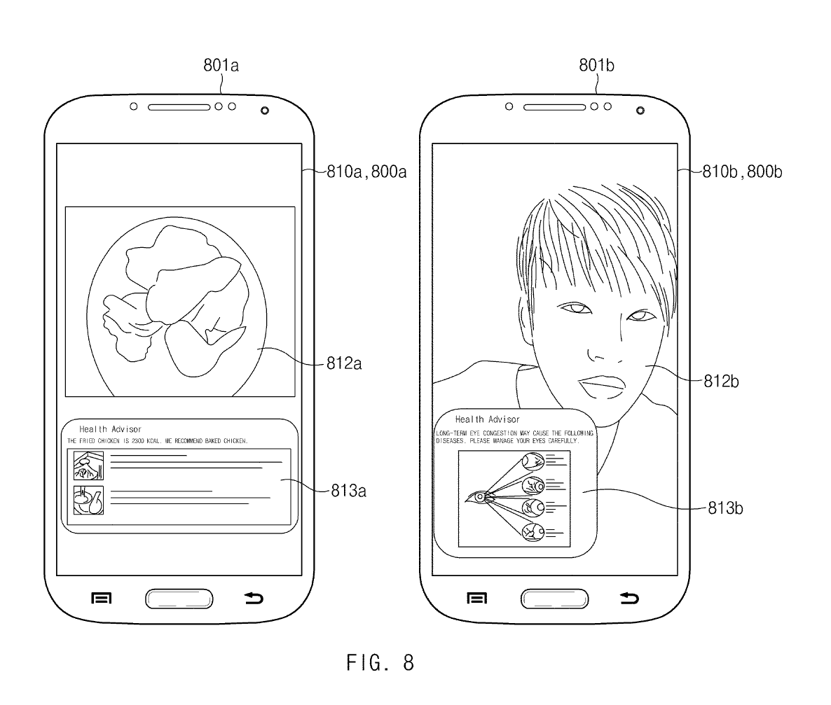

FIG. 8 illustrates aspects of an image viewer application, according to certain embodiments of this disclosure.

For example, a user may execute an image viewer application (e.g., Gallery) by using an electronic device 801a and may watch an image 812a obtained by capturing a food (e.g., fried chicken). A screen 800a including the image 812a obtained by capturing the food may be output in a display 810a of the electronic device 801a by the watching. Meanwhile, a health parameter may be pre-specified as “calorie”.

According to certain embodiments, the image 812a obtained by capturing the food and health content 813a provided by a server may be included in the execution screen 800a of the image viewer application. For the purpose of displaying the health content 813a on the execution screen 800a, for example, the electronic device 801a may perform the following operation.

According to some embodiments, the electronic device 801a may analyze correlations between “calories” as a pre-specified health parameter and the image 812a obtained by capturing the food, and may generate a specific query message depending on the analysis result. The electronic device 801a may transmit the query message to the server and may receive the health content 813a from the server in response to the query message.

As illustrated in FIG. 8, the health content 813a may include the “calorie” of the foods 812a displayed on the execution screen 800a of the image viewer application, and information about an alternative food recipe. The health content 813a may be output to a part of the execution screen 800a of the image viewer application. As such, when watching an image obtained by capturing the food, the user may receive health content associated with the “calorie” of the corresponding food together.

In a further example, the user may execute the image viewer application by using an electronic device 801b and may capture an image 812b (so-called “selfie”) obtained by photographing himself/herself. A screen 800b including the image 812b obtained by capturing the user may be output in a display 810b of the electronic device 801b. According to an embodiment, the health parameter may be pre-specified as an “eye state”, based on the health profile and the goal profile.

According to certain embodiments, the image 812b obtained by capturing the user and health content 813b provided by the server may be included in the execution screen 800b of the image viewer application. For the purpose of displaying the health content 813b on the execution screen 800b, for example, the electronic device 801b may perform the following operation.

According to some embodiments, the electronic device 801b may analyze correlations between “eye state” as a pre-specified health parameter and the image 812b obtained by capturing the user, and may generate a specific query message depending on the analysis result. The electronic device 801b may transmit the query message to the server and may receive the health content 813b from the server in response to the query message.

As illustrated in the non-limiting example of FIG. 8, the health content 813b may, according to certain embodiments, include medical information about the health state of eyes, which is grasped from the image obtained by capturing the user, for example, the bloodshot eye. The health content 813b may be output to a part of the execution screen 800b of the image viewer application. As such, when watching an image obtained by capturing himself/herself, the user may receive medical content associated with an “eye state” obtained from the corresponding image together.

According to various embodiments, in the electronic device 801b illustrated in FIG. 8, “eye state” as an example of a health parameter may not be specified in advance. For example, the processor of the electronic device 801b may designate a health parameter (“eye state”) based on the analysis result of an image, a text, or the like output to the display 810b. For example, the processor of the electronic device 801b may analyze the image, the text, or the like output to the display 810b and may dynamically extract and designate a health parameter (e.g., “eye state”, or the like) that is out of a normal range or a goal range, from the image, the text, or the like.

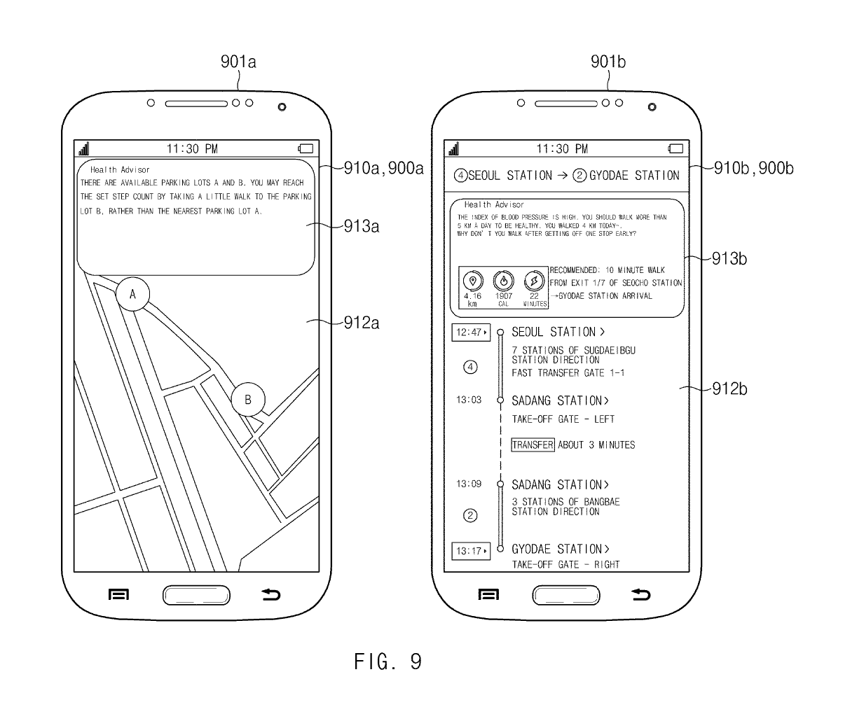

FIG. 9 illustrates aspects of a map application, according to certain embodiments of this disclosure.

In the non-limiting example of FIG. 9, a user may execute a map application by using an electronic device 901a and may search for an available parking lot in a specific area. A map and a location 912a of available parking lot candidates may be output to a display 910a of the electronic device 901a, by the searching. Meanwhile, a health parameter may be pre-specified as a “step count”.

According to certain embodiments, the map, the locations 912a of the available parking lot candidates, and health content 913a provided by a server may be included in an execution screen 900a of the map application. For the purpose of displaying the health content 913a on the execution screen 900a, for example, the electronic device 901a may perform the following operation.

According to some embodiments, the electronic device 901a may analyze correlations between “step count” as the pre-specified health parameter and the locations 912a of the available parking lot candidates, and may generate a specific query message depending on the analysis result. The electronic device 901a may transmit the query message to the server and may receive the health content 913a from the server in response to the query message.

As illustrated in FIG. 9, the health content 913a may, according to certain embodiments, include the locations 912a of the available parking lot candidates displayed on the execution screen 900a of the map application and an advice for achieving the goal step count. The health content 913a may be output to a part of the execution screen 900a of the map application. As such, when searching for the available parking lot, the user may receive health content associated with the goal step count together.

According to certain embodiments, the user may execute the map application (or a navigation application) by using the electronic device 901b and may, for example, search for a route from Seoul station to Gyodae station. A route 912b from Seoul station to Gyodae station may be output to a display 910b of the electronic device 901b by the searching. Meanwhile, a health parameter may be pre-specified as a “walking distance of the user”.

According to some embodiments, the route 912b from Seoul station to Gyodae station and health content 913b provided by the server may be included in an execution screen 900b of the map application. For the purpose of displaying the health content 913b on the execution screen 900b of the map application, for example, the electronic device 901b may perform the following operation.

According to certain embodiments, the electronic device 901b may analyze correlations between the “walking distance of the user” as the pre-specified health parameter and the route 912b from Seoul station to Gyodae station, and may generate a specific query message depending on the analysis result. The electronic device 901b may transmit the query message to the server and may receive the health content 913b from the server in response to the query message.

As illustrated in the non-limiting example of FIG. 9, the health content 913b may include advice for achieving the goal walking distance. The health content 913b may be output to a part of the execution screen 900b of the map application. As such, when searching for a route from Seoul station to Gyodae station, the user may receive health content associated with the goal walking distance.

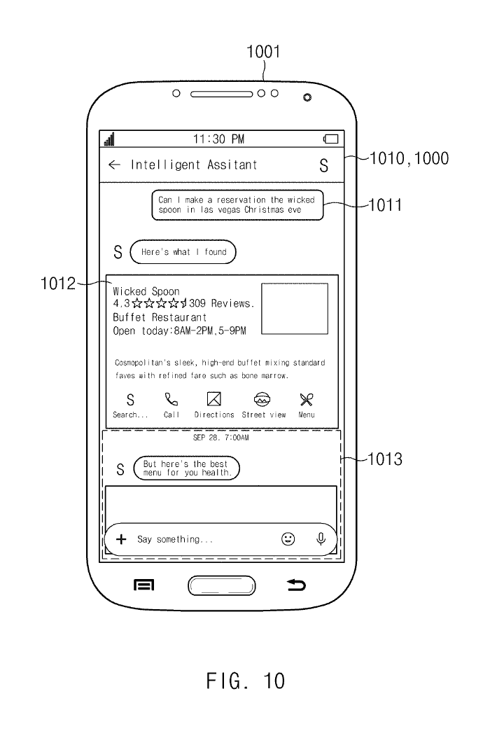

FIG. 10 illustrates aspects of an interactive application, according to certain embodiments.

In the non-limiting example of FIG. 10, a user may execute an interactive application (e.g., a personal assistant application, a speech recognition application, a messenger application in which a chatbot is embedded, or the like) to which artificial intelligence is applied, by using an electronic device 1001. The user may make a request 1011 for “reservation for Wicked Spoon in Las Vegas on Christmas Eve” to the interactive application in the form of voice or text. The interactive application may provide the user with “information 1012 about Wicked Spoon”, in response to the request 1011 of the user. Meanwhile, a health parameter may be pre-specified as a “body fat rate”.

According to an embodiment, the request 1011 of the user, a first response 1012 of the interactive application, and health content 1013 provided by a server may be included in an execution screen 1000 of the interactive application. For the purpose of displaying the health content 1013 on the execution screen 1000, for example, the electronic device 1001 may perform the following operation.

According to an embodiment, the electronic device 1001 may analyze correlations between “body fat rate” as the pre-specified health parameter and the “information 1012 about Wicked Spoon”, and may generate a specific query message depending on the analysis result. The electronic device 1001 may transmit the query message to the server and may receive the health content 1013 from the server in response to the query message.

As illustrated in FIG. 10, according to certain embodiments, the health content 1013 may include a menu suggestion, which is provided by “Wicked Spoon” and which is most suitable for the user's body fat rate. The health content 1013 may be output to a part of the execution screen, as one message in the interactive application. As such, when receiving information about a specific restaurant, the user may receive a recommendation together with a menu selected in consideration of the user's “body fat rate”.

According to some embodiments, an electronic device may include a display, a processor, a communication circuit establishing communication with a server, and a memory storing a specified application. The memory may store an instruction that, when executed, causes the processor to output an execution screen of the specified application to the display, in response to a launching of the specified application, to analyze correlations between a specified health parameter and content included in the execution screen, to generate a specified query message depending on the analysis result, to transmit the query message to the server, to receive a response content of the query message from the server, and to output the response content to at least part of the execution screen.

According to some embodiments of this disclosure, the memory may further store a health profile including a first group of heath parameters and a goal profile including a second group of heath parameters being a goal value of the first group of heath parameters. The instruction may cause the processor to further specify the specified health parameter depending on a comparison result between the first group of heath parameters and the second group of heath parameters.

In at least one embodiment according to this disclosure, the instruction may cause the processor to extract a common health parameter from the first group of heath parameters and the second group of heath parameters and, if a difference between a value of the common health parameter included in the health profile and a value of the common health parameter included in the goal profile is out of a specified range, to specify the common health parameter as the specified health parameter.

According to certain embodiments, the instruction may cause the processor to dynamically specify the specified health parameter based on the content included in the execution screen.

According to some embodiments, the specified health parameter may include at least one of a step count, a heart rate, an electrocardiogram (ECG), a blood pressure, glucose, a weight, a body fat, oxygen saturation, a body temperature, or skin conductivity.

According to at least one embodiment, the electronic device may further include a biosensor collecting biometric information corresponding to the specified health parameter.

According to certain embodiments, the electronic device may further include a short range communication module establishing connection to an external device. Biometric information corresponding to the specified health parameter may be collected from the external device.

According to at least one embodiment, the response content may include information associated with the specified health parameter.

According to certain embodiments, the information associated with the specified health parameter may include medical information about the specified health parameter.

According to some embodiments, the information associated with the specified health parameter may include at least one of nutrition information or exercise information for improving the specified health parameter.

According to some embodiments, a software developer's kit (SDK) for executing the instruction may be installed in the specified application.

According to at least one embodiment, the specified application includes at least one of a web browser application, an image viewer application, a messenger application in which a chatbot is embedded, a speech recognition application, or a map application.

According to certain embodiments, a health content providing method of an electronic device may include outputting an execution screen of the specified application in response to a launching of the specified application, analyzing correlationship between a specified health parameter and content included in the execution screen, generating a specified query message depending on the analysis result, transmitting the query message to a server, receiving a response content of the query message from the server, and outputting the response content to at least part of the execution screen.

According to some embodiments, the electronic device may store a health profile including a first group of heath parameters and a goal profile including a second group of heath parameters being a goal value of the first group of heath parameters. The method may further include specifying the specified health parameter depending on a comparison result between the first group of heath parameters and the second group of heath parameters.

According to certain embodiments of this disclosure, the specifying of the specified health parameter may include extracting a common health parameter from the first group of heath parameters and the second group of heath parameters and, if a difference between a value of the common health parameter included in the health profile and a value of the common health parameter included in the goal profile is out of a specified range, specifying the common health parameter as the specified health parameter.

According to at least one embodiment, the specified health parameter may include at least one of a step count, a heart rate, an electrocardiogram (ECG), a blood pressure, glucose, a weight, a body fat, oxygen saturation, a body temperature, or skin conductivity.

According to some embodiments, the method may further include collecting biometric information corresponding to the specified health parameter from an embedded biosensor or an external device.

According to at least one embodiment, the response content may include information associated with the specified health parameter.

According to certain embodiments, the information associated with the specified health parameter may include medical information associated with the specified health parameter.

According to some embodiments, the information associated with the specified health parameter may include at least one of nutrition information or exercise information for improving the specified health parameter.

According to some embodiments, the specified application may include at least one of a web browser application, an image viewer application, a messenger application in which a chatbot is embedded, a speech recognition application, or a map application.

The term “module” used in this disclosure may represent, for example, a unit including one or more combinations of hardware, software and firmware. The term “module” may be interchangeably used with the terms “unit”, “logic”, “logical block”, “component” and “circuit”. The “module” may be a minimum unit of an integrated component or may be a part thereof. The “module” may be a minimum unit for performing one or more functions or a part thereof. The “module” may be implemented mechanically or electronically. For example, the “module” may include at least one of an application-specific IC (ASIC) chip, a field-programmable gate array (FPGA), and a programmable-logic device for performing some operations, which are known or will be developed.

At least a part of an apparatus (e.g., modules or functions thereof) or a method (e.g., operations) according to various embodiments may be, for example, implemented by instructions stored in a computer-readable storage media in the form of a program module. The instruction, when executed by a processor (e.g., the processor 120), may cause the one or more processors to perform a function corresponding to the instruction. The computer-readable storage media, for example, may be the memory 130.

A computer-readable recording medium may include a hard disk, a floppy disk, a magnetic media (e.g., a magnetic tape), an optical media (e.g., a compact disc read only memory (CD-ROM) and a digital versatile disc (DVD), a magneto-optical media (e.g., a floptical disk)), and hardware devices (e.g., a read only memory (ROM), a random access memory (RAM), or a flash memory). Also, a program instruction may include not only a mechanical code such as things generated by a compiler but also a high-level language code executable on a computer using an interpreter. The above hardware unit may be configured to operate via one or more software modules for performing an operation according to various embodiments, and vice versa.

A module or a program module according to various embodiments may include at least one of the above elements, or a part of the above elements may be omitted, or additional other elements may be further included. Operations performed by a module, a program module, or other elements according to various embodiments may be executed sequentially, in parallel, repeatedly, or in a heuristic method. In addition, some operations may be executed in different sequences or may be omitted. Alternatively, other operations may be added.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. An electronic device comprising:
a display;
a processor;
a communication circuit configured to establish communication with a server; and
a memory configured to store a specified application,
wherein the memory stores an instruction that, when executed, causes the processor to:
output an execution screen of the specified application to the display, in response to a launching of the specified application;
analyze a correlation between a specified health parameter and content included in the execution screen to obtain a result;
generate a specified query message depending on the analysis result;
transmit the specified query message to the server;
receive a response content of the query message from the server; and
output the response content to at least part of the execution screen.

2. The electronic device of claim 1, wherein the memory further stores a health profile including a first group of heath parameters and a goal profile including a second group of heath parameters being a goal value of the first group of heath parameters, and
wherein the instruction further causes the processor to:
specify the specified health parameter depending on a comparison result between the first group of heath parameters and the second group of heath parameters.

3. The electronic device of claim 2, wherein the instruction further causes the processor to:
extract a common health parameter from the first group of heath parameters and the second group of heath parameters; and
if a difference between a value of the common health parameter included in the health profile and a value of the common health parameter included in the goal profile is out of a specified range, specify the common health parameter as the specified health parameter.

4. The electronic device of claim 1, wherein the instruction further causes the processor to:
dynamically specify the specified health parameter based on the content included in the execution screen.

5. The electronic device of claim 1, wherein the specified health parameter includes at least one of a step count, a heart rate, an electrocardiogram (ECG), a blood pressure, glucose, a weight, a body fat, oxygen saturation, a body temperature, or skin conductivity.

6. The electronic device of claim 1, further comprising:
a biosensor configured to collect biometric information corresponding to the specified health parameter.

7. The electronic device of claim 1, further comprising:
a short range communication module configured to establish connection to an external device,
wherein biometric information corresponding to the specified health parameter is collected from the external device.

8. The electronic device of claim 1, wherein the response content includes information associated with the specified health parameter.

9. The electronic device of claim 8, wherein the information associated with the specified health parameter includes medical information about the specified health parameter.

10. The electronic device of claim 8, wherein the information associated with the specified health parameter includes at least one of nutrition information or exercise information for improving the specified health parameter.

11. The electronic device of claim 1, wherein a software developer's kit (SDK) for executing the instruction is installed in the specified application.

12. The electronic device of claim 1, wherein the specified application includes at least one of a web browser application, an image viewer application, a messenger application in which a chatbot is embedded, a speech recognition application, or a map application.

13. A health content providing method of an electronic device, the method comprising:
outputting an execution screen of a specified application in response to a launching of the specified application;
analyzing a correlation between a specified health parameter and content included in the execution screen to obtain a result;
generating a specified query message depending on the analysis result;
transmitting the query message to a server;
receiving a response content of the query message from the server; and
outputting the response content to at least part of the execution screen.

14. The method of claim 13, wherein the electronic device stores a health profile including a first group of heath parameters and a goal profile including a second group of heath parameters being a goal value of the first group of heath parameters,
further comprising:
specifying the specified health parameter depending on a comparison result between the first group of heath parameters and the second group of heath parameters.

15. The method of claim 14, wherein the specifying of the specified health parameter includes:
extracting a common health parameter from the first group of heath parameters and the second group of heath parameters; and
if a difference between a value of the common health parameter included in the health profile and a value of the common health parameter included in the goal profile is out of a specified range, specifying the common health parameter as the specified health parameter.

16. The method of claim 13, further comprising:
collecting biometric information corresponding to the specified health parameter from an embedded biosensor or an external device.

17. The method of claim 13, wherein the response content includes information associated with the specified health parameter.

18. The method of claim 17, wherein the information associated with the specified health parameter includes medical information associated with the specified health parameter.

19. The method of claim 17, wherein the information associated with the specified health parameter includes at least one of nutrition information or exercise information for improving the specified health parameter.

20. The method of claim 13, wherein the specified application includes at least one of a web browser application, an image viewer application, a messenger application in which a chatbot is embedded, a speech recognition application, or a map application.
